(19) United States
(12) Patent Application Publication
(10) Pub. No.: US 2002/0193836 A1
(43) Pub. Date: Dec. 19, 2002

(54) Leads for the treatment of patients with CHF

(76) Inventor: John A. Schmidt, Durango, CO (US)

Correspondence Address: GOTTLIEB RACKMAN & REISMAN PC, 270 MADISON AVENUE, 8TH FLOOR, NEW YORK, NY, 100160601

(21) Appl. No.: 10/135,061

(22) Filed: Apr. 29, 2002

Related U.S. Application Data
(60) Provisional application No. 60/287,145, filed on Apr. 27, 2001.

(51) Int. Cl. A61N 1/362

(52) U.S. Cl. 607/9

(57) ABSTRACT

A multi-electrode lead is provided for a cardiac device, the lead having anywhere from three to 258 electrodes. The lead is preformed to a particular shape selected so that when implanted, its electrodes are in contact with a particular cardiac tissue to sense cardiac activity and to provide CHF pacing. The lead can have a V-shape to stimulate the septal wall of the right ventricle, it could be spiral to simulate the conduction of a polarization wave of a sinus Purkinje cell system.

### RELATED APPLICATIONS

This application claims priority to provisional application Ser. No. 60/287,145 filed Apr. 27, 2001, incorporated herein by reference.

The subject matter of this application is also related to the following commonly assigned co-pending patent applications, incorporated herein by reference:

Cardiac Electrode Catheter and Method of Manufacturing Same, Ser. No. 09/761,333, filed Jan. 16, 2001, now______;

A Multi-Electrode Cardiac Lead Adapter with Multiplexer, Ser. No. 10/0,62,138 filed Feb. 1, 2002, now______;

System and Method for Treatment of Congestive Heart Failure Utilizing a Single Lead Multi-Focal Cardiac Pulse Generator System, Ser. No. 10/075,808 filed Feb. 13, 2002, now______;

Cardiac Stimulation Apparatus and Method for Treatment of Atrial Fibrillation, Ser. No.______ filed Apr. 26, 2002; and

Method and apparatus for Determining Spatial Relation of Multiple Impantable Electrodes Ser. No.______ filed Apr. 25, 2002.

### BACKGROUND OF THE INVENTION

a. Field of Invention

This application pertains to pacemaker leads particularly suited for the treatment of cardiac diseases such arrhythmia, catastrophic heart failure and other similar procedures requiring the application of electrical pulses. Each lead consists of a multi-electrode array that has been specifically shaped to fit within the patient's heart in such a manner that its electrodes are distributed at various strategic locations, dependent on the desired treatment. A plurality of lead configurations are presented, each configuration being suitable for a particular type of cardiac therapy or procedure.

b. Description of the Prior Art

Cardiac diseases are due to complicated pathological conditions and seriously affect the lives of millions of patients. The For example, congestive Heart Failure (CHF) currently affects 5 million Americans, 6.5 million Europeans, and a worldwide total of 22 million people. There are approximately 550,000 new cases diagnosed annually in the U.S. and CHF is the underlying cause or major contributing factor in more than 300,000 U.S. deaths each year. The median survival time in the U.S., following the onset of CHF is 1.7 years for men and 3.2 years for women.

Extensive clinical research has shown that properly delivered single and multi-site septal pacing in the right ventricle (RV) can re-synchronize left ventricular contraction in patients with moderate to severe CHF, improving their quality of life and potentially extending their life span. It has been reported that the improvement in cardiac output possible with right ventricular re-synchronization therapy is comparable to, or better than that achieved using bi-ventricular (Bi-V) re-synchronization pacing therapy, but without the complications inherent to the Bi-V approach.

The heart is a mechanical pump that is stimulated by electrical impulses. The mechanical action of the heart results in the flow of blood. During a normal heartbeat, the right atrium (RA) fills with blood from the returning veins. The RA then contracts and this blood is moved into the right ventricle (RV). When the RV contracts it pumps that blood to the lungs. Blood returning from the lungs moves into the left atrium (LA), and after LA contraction, is pumped into the left ventricle (LV) which then pumps it throughout the body. Four heart valves keep the blood flowing in the proper directions.

The electrical signals that drives this mechanical contraction start in the sino-atrial node, a collection of specialized heart cells in the right atrium which automatically depolarize (change their voltage potential). This depolarization wave front passes across all the cells of both atria and results in atrial contraction. When the advancing wave front reaches the A-V node it is delayed so that the contracting atria have time to fill the ventricles. The depolarizing wave front then passes over the ventricles causing them to contract and pump blood to the lungs and body. This electrical activity occurs approximately 72 times a minute in a normal individual and is called normal sinus rhythm.

CHF is caused by a number of different etiologies. The main effect of CHF is that it reduces the efficiency of the heart causing it to work harder to maintain adequate blood flow through the body. As a result, over time, the muscle fibers of the heart enlarge and the entire heart gets bigger. Frequently, the heart can increase from two to five times its normal size. In fact, this enlargement is one of the symptoms of CHF.

Treatment for CHF varies and may includes drug, bi-ventricular pacing, alternate site pacing, bifocal pacing, or a combination of at least some of these regimes.

Drug therapy includes the administration of diuretics to reduce plasma volume. (By decreasing the total volume of blood the heart doesn't work as hard. Veno- and vaso-dilators are used to reduce cardiac preload and after load. (Likewise, by increasing the size of the ‘pipes’ (veins and arteries) of the body it is easier for the heart to pump.) Inotropic agents like digitalis are used to enhance cardiac contractility. The drugs are intended to reduce the workload on the heart or to increase the strength of cardiac contraction. While drug therapy is a valuable tool in controlling heart failure, it is ineffective or intolerable for many patients. For example, a study showed that nearly 9% of all patients taking Candesartan and enalapril suffered adverse effects. In another study, mibefradil (a calcium channel blocker) was tested and it was concluded that the drug had no favorable effects in patients with CHF. None of the drug regimens are viable, long-term treatments for CHF. Moreover, it has been estimated that about 30% of patients exhibit poor compliance and never obtain prescriptions refills.

Bi-ventricular cardiac resynchronization pacing devices to treat CHF have been tested and introduced in Europe. These devices resynchronized the left ventricular contraction by pacing epicardially via a lead inserted through the great cardiac vein (GCV). The intent was to have one pacemaker lead over the LV along with another lead inside the RV and then, based on the timing of the normal electrical signals, caused the pacemaker to synchronize the left and right contractions.

Re-synchronization therapy is at the early stages of testing by the medical community, and is associated with numerous complications including lead dislodgements from the GCV, coronary sinus dissection, cross talk between the leads, and left phrenic nerve stimulation. In addition, the implantation of the lead into the LV also takes from 1.5-5 hours. Consequently, these and similar left-sided resynchroni-zation devices are adopted very slowly and ultimately their success may be severely limited by the technical drawbacks of this approach.

RVOT (right ventricular outflow track) pacing has been compared to right ventricular apical pacing in several studies which included CHF patients. A number of different cardiac parameters were measured and all of the metrics of left ventricular function were greatly increased with RVOT pacing as opposed to right ventricular apical pacing. Several items were noted from these studies. First, the patients who benefitted the most from RVOT pacing were those with the lowest baseline cardiac index, i.e., those patients with the most severe CHF. Second, the improvement in cardiac performance rivaled that achieved with left ventricular or Bi-V pacing. But there was a large spread in the reported data which may be due to not having located the optimal pacing site in the RVOT.

Multi-focal (2 Site) RV Pacing. There are also clinical reports indicating that pacing from two sites in the RV produces excellent results for patients suffering from heart failure. These bi-focal pacing studies suggest that this pacing modality may rival the results obtained with left ventricular or bi-ventricular pacing, but without the complications and difficulties associated with those techniques.

Direct His-bundle pacing in heart failure patients has also been demonstrated as a possible CHF therapy. Permanent direct His-bundle pacing leads (standard pacemaker leads) were implanted in 12 out of 18 patients, aged 69±10 years, who had a history of chronic atrial fibrillation, dilated cardiomyopathy, and normal activation (i.e., QRS less than or equal to 120 msec) The study shows that this type of therapy resulted in significant improvement in cardiac output. This study is of particular interest since it suggests that direct His-bundle pacing, entirely from the right side of the heart, can make significant improvements in cardiac function in the large majority of severe CHF patients with intact conduction systems.

Finally it is possible to use the conformal multi-electrode leads in the RA and RV in conjunction with a bi-ventricular lead in the great cardiac vein. This approach combines the advantages of direct left ventricular free-wall pacing with the capability of the lead system to pick optimized pacing sites and AV delays on the right side of the heart.

### OBJECTIVES AND SUMMARY OF THE INVENTION

Alternate right side pacing, right side bi-focal pacing, and right side direct His-bundle pacing have demonstrated the clinical efficacy of a multi-focal right side lead system for the treatment of CHF. The lead and system described is capable of delivering ail of these pacing therapies. It is also be capable of stimulating at more than two (preferably 16) locations in the RV. One particularly compelling advantage of this would be in patients who have impaired contractility along the septal wall due to past myocardial infarctions. Coordinated multi-site pacing, optimized to the patient, results in a left ventricular synchronized action potential that compensates for the inability of a damaged left ventricle to contract when stimulated either by the normal conduction system or by a compromised conduction system.

The right-sided CHF cardiac lead and system described here will use a multi-focal pacing lead placed in the RV. The system will distribute multiple electrodes in contact with the myocardium of the right ventricle with the electrodes spaced at predetermined intervals along the lead. This ventricular lead is pre-shaped to deploy more than 2 and preferably sixteen electrodes strategically along the entire septal wall from a point near or in the right ventricular outflow tract (RVOT) to the apex of the ventricle.

In view of the disadvantages of the prior art, it is an objective of the present invention to develop a right-sided cardiac resynchronization system which utilizes an optimized multi-focal pacing technique in the right ventricle. The multi-focal pacing system described in this disclosure is ideally suited to addressing these difficulties. The multiple electrodes on the lead are spread out along the right ventricular septum. One or more of these electrodes are implanted so that they reside at, or very near the optimal pacing site(s). The physician can determine the optimal pacing site(s) by pacing off each of the electrodes in turn, or can allow the system to automatically determine the optimal site(s) based on the QRS duration or some other cardiac metric. This system will take the guesswork out of RV placement and improve the consistency of RV alternate site pacing.

The system employs a lead technology that makes it possible to place multiple pacing and sensing electrodes in contact with the myocardium in the RV. The lead is pre-shaped to deploy up preferably sixteen electrodes along the entire septal wall from the RVOT to the apex of the RV. A second more conventional lead can be placed in the right atrium to provide proper atrial sensing. The lead will be deployed using a standard stylet or with a guide catheter. Leads can be customized to accommodate variations in patient anatomy.

The invention pertains to an implantable cardiac sensing and stimulation system with the appropriate sensing and pacing circuitry (pulse generator) and a multi-electrode lead attached to the stimulator and shaped for insertion into one (or more) chambers of the heart. The sensing and stimulation system is adaptable and can sense intrinsic cardiac activity and generate a stimulation pulse responsive to that electrical activity.

It will be possible to deliver a number of different pacing therapies for the treatment of CHF with this system. The simplest use will involve choosing a single electrode from the many electrodes on the lead and pacing through that single electrode to improve cardiac output. Alternatively, pacing through two or more electrodes simultaneously, or through a number of electrodes sequentially can be used to treat different pathologies. The advantage of a multi-electrode lead is that having many electrodes minimizes the problem of finding this optimal pacing site (sweet spot). The physician can determine the optimal pacing electrode manually at the time of implantation or allow the pacing system itself to automatically determine the optimal pacing electrode. Implantation time for this system will be comparable to that of a standard pacemaker, on the order of 10 to 20 minutes, as opposed to up to several hours for bi-ventricular leads.

The system will improve on this by allowing for pacing from additional sites if desired, by optimizing the selection of the pacing sites, and by coordinating the pacing pulse delay to each electrode in order to maximize cardiac output for each individual patient.

In the present invention, a multi-electrode lead is used to provide CHF therapy, the lead including between two and up to 128 independent electrodes. A lead of this type is described in commonly assigned application Ser. No 09/761,333 referenced above and incorporated herein by reference. In the preferred embodiment the lead has 16 electrodes. Each electrode on the lead can be used for both sensing electrical signals associated with the electrical activity of the heart and delivering electrical pulses to the heart. The delivery of pulses is optimized for bradycardia pacing and multi-site stimulation for congestive heart failure, as described more fully below.

The lead can be placed so that it extends into one or more chambers of the heart. Briefly, the lead is preferably made up of an external biocompatible thermoplastic polymer tube such as polyurethane, and its electrodes extend circumferentially around the tube and are connected to internal conductors extending through the tube. Each conductor is coated with an electrical insulator, thereby insuring the electrical isolation of each electrode. By choosing the appropriate polymer tubing the lead can be thermally shaped to conform to virtually any configuration without the aid of a pre-shaped wire or shape memory alloy wire. Thus the lead can be fully constructed and later shaped to fit a given chamber of the heart or a particular patient's pathology. One embodiment uses a coil shaped catheter that places electrodes around an entire chamber of the heart. This embodiment allows complete sensing and stimulating control of the entire chamber. The electrodes can be spaced along the lead, with different spacing being provided for different applications.

Preferably, a separate lumen extends through the length of the tubing and is reserved for a stylet. The stylet is used to implant the lead into the heart in the same manner as a standard pacemaker lead. The stylet straightens the lead from its preformed shape as it passes through the veins and, when removed, allows the lead to deploy into the selected chamber of the heart.

What makes this lead particularly useful for the treatment of CHF is that the size, spacing, and final geometry of the lead can be customized for the treatment of CHF. For this purpose, the lead is placed in the RV (and the RA), with multiple electrodes deployed to provide a choice of pacing sites at locations best suited to multi-focal control of the failing heart. The lead is then connected to a pacer which includes a software-controlled microprocessor. The software determines the pacing for each of the sites in a manner optimized to the patient and heart condition according to current research and standard metrics of cardiac output.

In accordance with this invention, treatment of CHF is accomplished by sending out a pulse from a preselected electrode (E1), measuring cardiac output metrics and comparing these metrics to baseline data. A pulse is then sent through a different electrode, cardiac metrics measured and compared to baseline and the respective electrode E1. This process is continued until all electrodes have been tested. The electrode with the best metric then becomes the pacing electrode. This is one brute force method.

Multi-site pacing is accomplished by selecting a pair of electrodes and pacing both of them simultaneously, measuring cardiac metrics and comparing these metrics to baseline. Then a second pair of electrodes, are paced, metrics measured and compared to baseline and the results from the first pair. Next, a third pair of electrodes are paced and so on in a round robin process. In this way a data base of cardiac metrics and electrodes paced can be built up and the system can select the best pair of electrodes to pace from.

Moreover, the process does not have to be limited to two electrodes. Three electrodes can be paced simultaneously and then using a similar round robin system. Cardiac parameters measured from each set, and the data base built up to determine which electrodes to pace simultaneously. Similarly, sequential pacing from 2, 3, 4, or more electrodes can be accomplished with this methodology.

### BRIEF DESCRIPTION OF THE DRAWINGS

FIG. 1. shows a somewhat diagrammatic cross-sectional view of a patient's heart with a prior art two electrode lead;

FIG. 2. shows a partial side view of showing details of one of the basic construction of one of the electrodes of a multi-electrode lead constructed in accordance with this invention;

FIG. 2A shows an alternate construction of the electrodes in accordance with this invention;

FIG. 3 shows an orthogonal view of a multi-electrode lead constructed in accordance with the invention;

FIG. 4 shows a preshaped RVOT lead extending into the RV and connected to a pacemaker;

FIG. 5 shows a partial cross sectional view of a patient's heart with the RVOT lead extending into the RV;

FIG. 6 shows a somewhat diagrammatic representation of the septum fibers in a region below the RVOT and an optimal point for pacing the ventricle;

FIG. 7 shows a somewhat diagrammatic representation of the septum fibers in the region below the RVOT with a plurality of electrodes arranged along the septal wall;

FIG. 8 shows a multi-electrode lead shapes as a spiral and disposed in the right ventricular chamber;

FIG. 9 shows a multi-electrode lead with a loop disposed in the right atrium for stimulating the bundle of His;

FIG. 9A shows an enlarged portion of the loop of FIG. 9;

FIG. 10 shows a flow chart for the selection of electrodes to be used for sensing and pacing;

FIG. 11 shows a block diagram illustrating the three-dimensional positioning of the lead.

### DETAILED DESCRIPTION OF THE INVENTION

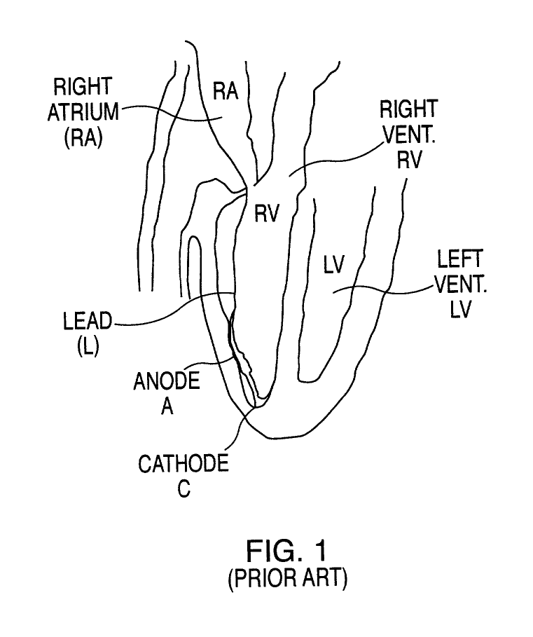

FIG. 1 shows a standard bipolar pacing lead L that is implanted through the superior vena cava (not shown) into the right atrium RA and right ventricle RV. The lead is terminated with tip electrode that is positioned near the apex of the right ventricle and acts as a cathode electrode C. The lead is further provided with an anode electrode A disposed at some distance away from the cathode electrode C.

A pacing pulse is applied to the tip or cathode electrode to cause a retrograde contraction of the cardiac muscles that propagates in an opposite direction from the normal contraction associated with a sinus heart beat. The anode provides the return path for the pacing pulse to the pulse generator (shown in FIG. 4).

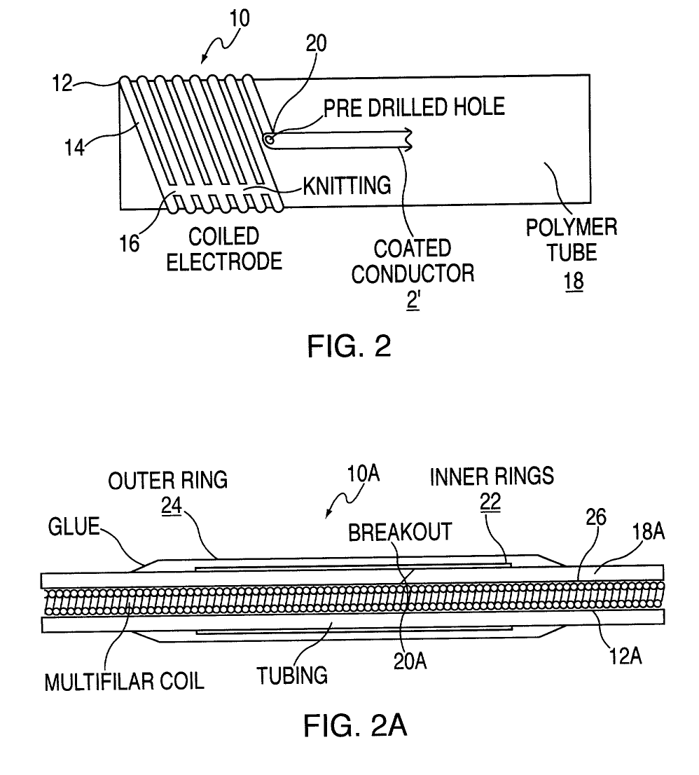

In the present invention a multiple electrode lead is used having anywhere from four to 256 or even more electrodes. As shown in FIG. 2, typically, each electrode 10 is formed of a coiled wire 12 formed of individual loops 14. Preferably the loops are coupled together, for example, by welding, knitting, or, as shown in FIG. 2, by a bar 16, to increase the mechanical strength and dimensional stability of the electrode 10.

The electrode is placed on a non-conductive tube 18 made of a polymer such as polyurethane or other similar material. The wire 12, which is made of a metallic material, extends through a hole 20 made in the tube 18, and then through the tube the distal end of the lead. Inside the tube, the wire 12 is covered or coated with an insulating jacket 22 made of polymer (i.e., polyurethane, silicone, fluropolymer) or any other similar insulator.

Details of making the lead in this manner are disclosed in co-pending application Ser. No. 09/761,333 identified more fully above, and incorporated herein by reference. This particular lead structure has several advantages. The pre-drilled holes define unequivocally and precisely the position of each electrode 10. These positions and spacing may be selected so that the electrodes are positioned along the septal wall along the right ventricle in a particular configuration as disclosed in more detail below. The electrodes extend circumferentially around the tube 18 thereby insuring that if the tube is in close proximity to any cardiac tissues, the respective electrodes are in contact with the respective tissues.

Standard prior art leads have a metal tip which is pushed into the cardiac muscle and over time can penetrate and injure the muscle. Circumferential electrodes do not have sharp tips and therefore will not injure the muscles.

FIG. 2A shows an alternate embodiment of an electrode 10A. This electrode 10A consists of two concentric rings: an inner ring 22 and an outer ring 24. The rings are disposed in a tube 18A. Inside the tube 18A there is a multi-filament cable 26. Cable 26 is formed of a plurality of insulated wires 12A, one for each electrode 10A. At each electrode 10A, one of the wires 12A is broken out through a hole 20A. The end of the wire is then crimped between the two rings 22,24. The wire 12A can also be spot-welded to the rings. The two rings can be glued or otherwise connected to each other in a known matter.

As in the embodiment of FIG. 2, the holes 20A are drilled within the tube 18A at the desired locations for the electrodes 10A. In the embodiment shown in FIG. 2A, the tube 12 is continuous. Alternatively, the tube can be cut at each electrode, the respective wire is broken out from the cable. And the tube parts can then be pushed together to meet under the electrode or reconnected in some other manner. This embodiment is less desirable if cable 26 has more than four wires because its pitch may cause it to fail due to mechanical fatigue.

Other means of making a multi-electrode lead may be used as well.

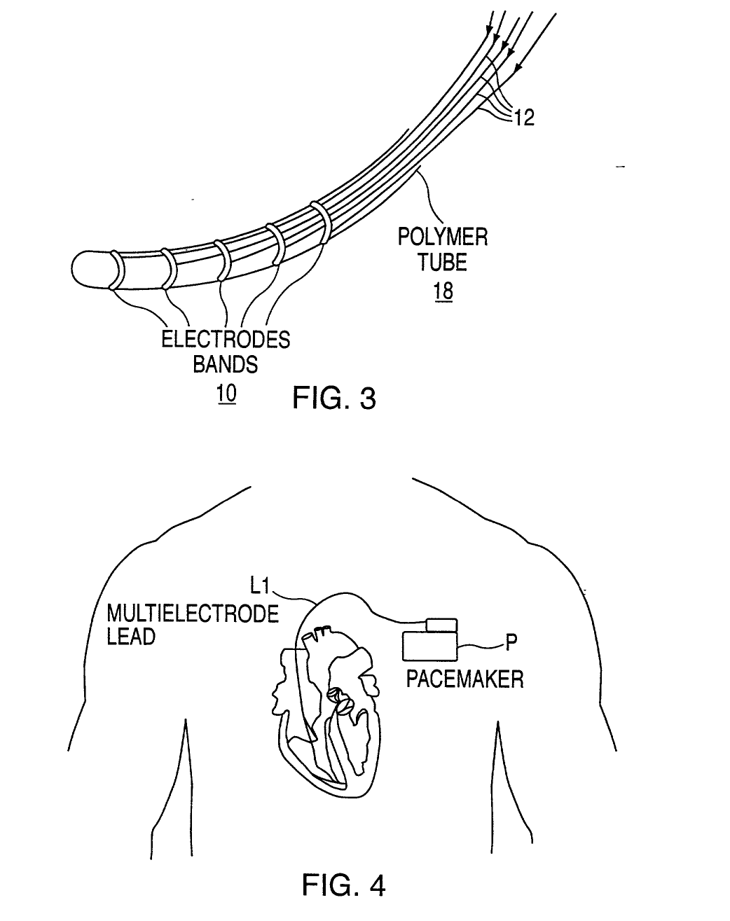

FIG. 3 shows an orthogonal sectional view of a lead L constructed in accordance with FIG. 2. The wires within the tube can be arranged to allow a stylet to be introduced into the tube to guide the lead during implantation.

FIG. 4 shows a cardiac therapy system adapted to treat CHF in accordance with this invention. Details of the system are found in application Ser. No. 10/075,808). The system includes a pacemaker P implanted in the normal manner and a multi-electrode lead L extending into the cardiac chambers as discussed above. The lead L is connected to the pacemaker P by a lead adapter as disclosed in application Ser. No. 10/062,138) identified more fully above, or by other similar means. The exact shape, size, number of electrodes and geometric configuration of the lead depends on the particular CHF therapy to be applied. In the following discussion, leads having several configurations are described, each lead being suitable for a particular type of therapy.

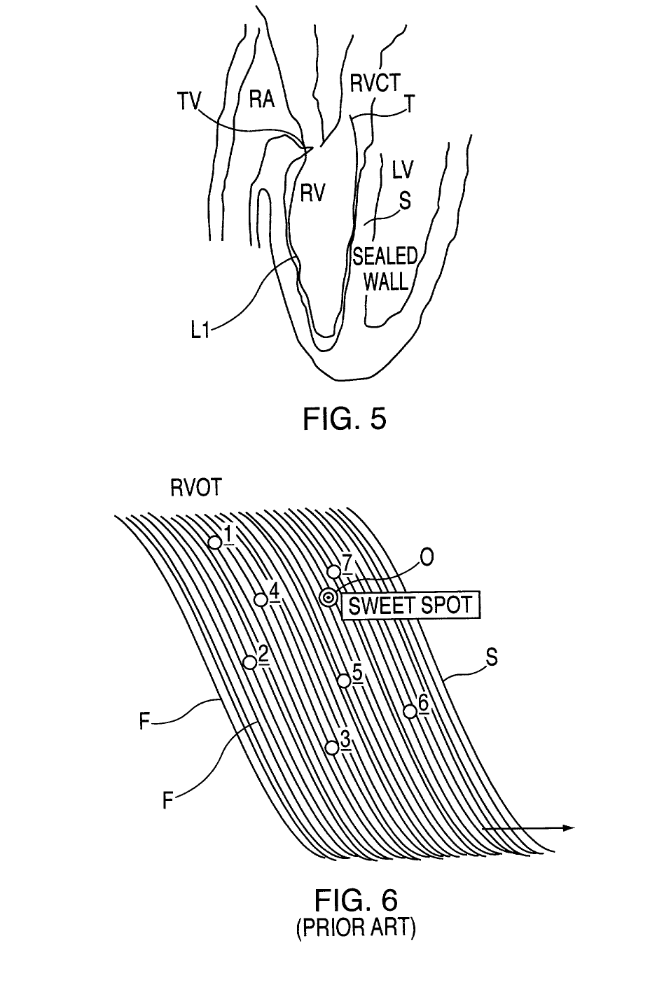

FIG. 5 shows a lead having a generally V-shaped configuration, in which the lead extends downward near the apex of the RV and then back up along the septum wall S with its tip T terminating in the RVOT. Because this lead passes between the RA and RV it must cross the tricuspid valves TV. Valve TV is unique in that it opens and closes in response to the contraction of the heart. Valve TV is totally passive and it is the flow of blood over the valve that determines whether it opens or closes. For the tricuspid valve, it means that the valve is “anchored” between the RA and RV by a set of tendons (chorda tendoneae, small thin cords) which extend down into the ventricle. The ends of these tendons are attached to papillary muscles (undulations of the inner ventricular wall). The tendons and muscles impart a trabeculated structure to the inside of the ventricle and the lead is designed to slip between the tendons and muscles in order to minimize rubbing and abrasion.

The tip T is anchored at the right ventricular outlet track. FIG. 6 shows a somewhat schematic representation of a region of the septal wall S with the fibers F oriented somewhat parallel to each other. Point O represents the optimal point from which the septum can be paced more effectively, and is referred to as the ‘sweet spot.’ Since this optimal point is not readily discernible and its location differs from patient to patient, normally a clinician has to use a trial-and-error method for detecting the optimal point. If the lead is not in the correct location the first time, i.e., adjacent to the point O, the clinician has to reposition it, pace the heart, and determine a new set of cardiac parameters. This process goes on the lead is implanted so that its respective electrode is located at an effective distance from the optimal point. For example, positioning the electrode to location 1 will not produce optimal cardiac output parameters, and neither will position 2. It has been reported that it takes a median of seven attempts with up to eighteen attempts to locate this sweet spot. FIG. 6 shows seven such attempts with attempt No. 7 being closest.

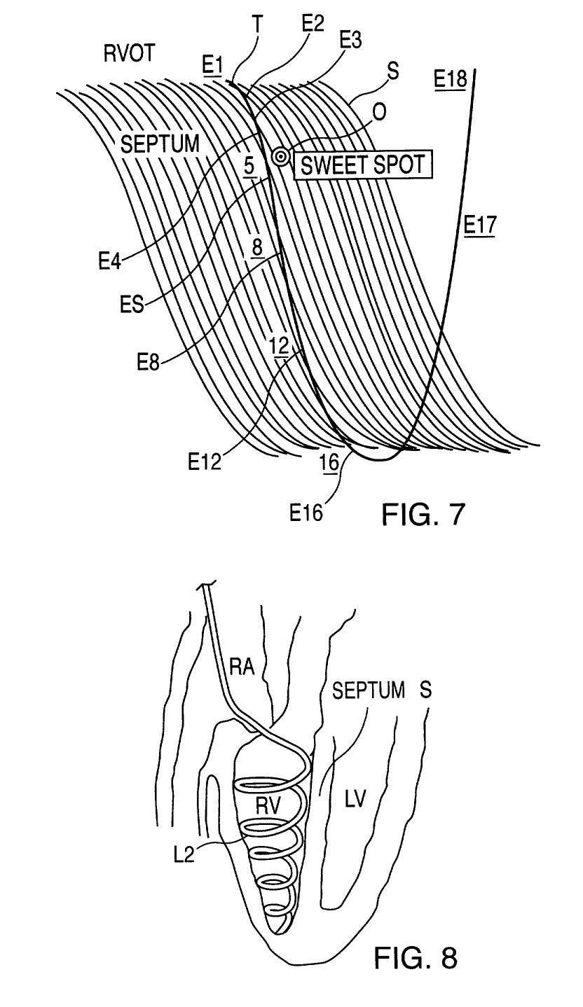

FIG. 7 shows a similar representation to the one in FIG. 6 showing a multi-electrode lead L arranged in a V-shaped configuration. In this configuration, the tip T of the lead L is disposed at the RVOT, with electrodes E1, E2 . . . E16, being positioned along the septal wall S as shown. Additional electrodes E17, E18, etc. are positioned along the outer wall of the right ventricle. Once the lead L is implanted, the electrodes known to be located somewhere near the optimal point O are tested in the standard manner, and the electrode closest to the optimal point O (in this case E5) is determined.

The lead L can now be used either as a standard ventricular pacing lead with the electrode E5 being used as the pacing electrode. Alternatively, as previously discussed, pacing pulses are provided in sequence to electrodes E5-E16 to mimic the propagation of normal polarization waveforms downwardly along the septal wall from the RVOT to the lower tip of the ventricle.

Alternatively, the electrodes E17 and E18 may also be excited to promote contraction of the whole ventricle.

FIG. 8 shows an alternate lead employment. In this embodiment, the lead L2 is a multi-electrode lead shaped like a spiral and deployed in the right ventricle and its tip T is terminated in the apex A. Some of the electrodes (not shown) are in contact with the septal wall S while other electrodes are in contact with the outer wall W of the right ventricle. This configuration is particularly useful for cardiac patients having some form of conduction problems in the Purkinje system. The Purkinje system includes specialized cells adapted to conduct electrical pulses faster then normal heart cells. The Purkinje cells are located to allow a depolarization wave front to travel down the septum and around the circumferential heart walls very quickly thereby causing the muscles of the heart to contract substantially simultaneously. A disruption of the Purkinje system cases abnormal cardiac contraction. The electrodes of lead L2 can be used to apply multi-focal pacing to the ventricle in a manner that imitates the natural sequence of the healthy Purkinje system.

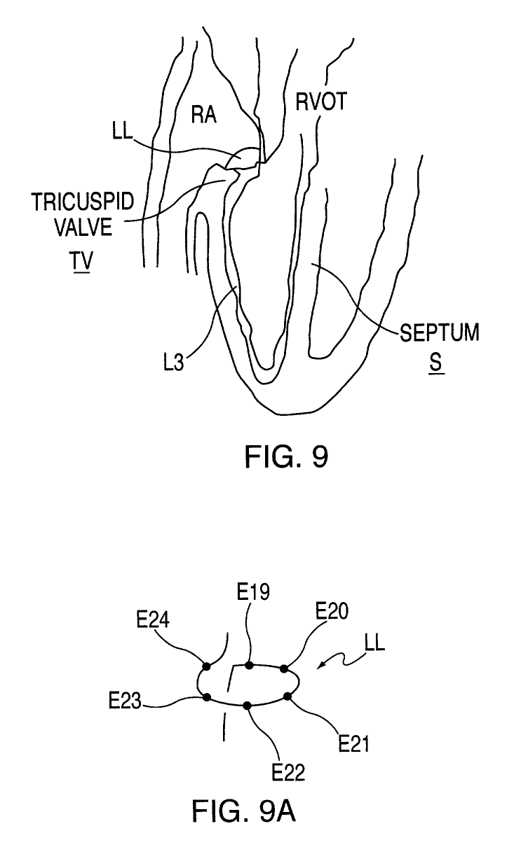

FIG. 9 shows another embodiment of the invention. In this embodiment, a generally V-shaped lead L3 is provided which has the same characteristics within the ventricle as lead L1 in FIG. 5 but in addition it also has a small loop LL disposed in the right atrium RA above the tricuspid valve TV. The purpose of this loop LL is to provide excitation to the bundle of His. Of course, the lower, or distal portion of the lead L3 can have any other shape as well, or it may be omitted.

The bundle of HIS is a small band of atypical cardiac muscle fibers that starts in the AV node and runs into the interventricular septum S. In this septum these fibers split into the left and right bundle branches which extend down into the two (left and right) ventricles. This bundle of cells propagates the atrial contraction to the ventricles. If a patient suffers from problems in the AV node and the bundle of HIS is intact, the lead L3 is used to pace the heart by pacing directly into the bundle of HIS. This type of pacing is advantageous because it produces a contraction very similar to a sinus contraction since it utilizes the normal conduction system of the heart. For this purpose, the loop LL is provided with a plurality of electrodes E19-E24.

In FIGS. 5-9A shows the preferred locations of the electrodes with respect to the walls and other features of the cardiac chambers for pacing to provide therapy for CHF. In order to insure that the electrodes are actual placed in this locations, consideration must be given to the spacing of these electrodes. For example, if a patient has a septal wall 40 mm in length the respective lead is constructed such that its tip is inserted into the right ventricular outflow tract (as shown) and its first electrode E1 is placed near the moderator band (approximately 20 mm from the tip T).

If all subsequent electrodes, proximal to this first electrode E1, are spaced at 4 mm intervals then for a straight lead (FIGS. 5, 7, 9) five additional electrodes can be placed on the septal wall. If the electrodes are spaced at 3 mm intervals then seven electrodes can be positioned for pacing. If the electrodes are spaced 2 mm apart then ten electrodes can be utilized in the remaining 20 mm of septal wall.

Since the spacing of the electrodes is also dependent on the size of the electrodes there is a minimum spacing interval for a given length of electrode. One of the advantages of the method for manufacturing the electrodes, as described in copending application Cardiac Electrode Catheter and Method of Manufacturing Same, Ser. No. 09/761,333, filed Jan. 16, 2001, is that the electrode size can be easily changed.

In the preferred embodiment of the lead the electrodes (shown in FIG. 2) consist of 4 turns of 0.076 mm diameter wire with a total length of 0.304 mm (4×0.076). If the spacing between the electrodes is the same length as the electrodes, then for a 20 mm length of septal wall, the maximum number of electrodes that can be placed into a 20 mm length is 32 (20 mm divided by 0.608).

Similarly, the minimum length of an electrode is 0.152 mm and if the gap between electrodes is equivalent to the length of the electrodes then the maximum number of electrodes that can be placed along the septal wall is 65 (20 divided by 0.304).

Thus from 1 to 65 independent electrodes are placed in direct contact with the septum by controlling the spacing and size of the electrodes. For patients with different heart sizes, the number of electrodes, as well as their size and spacing is changed accordingly.

The electrodes not designated to be adjacent to the septal wall may have the same spacing, or a different spacing. For example, the electrodes for stimulating the free walls of the right ventricular chamber may be spaced at 15 mm. The electrodes on loop LL (FIG. 9A) for stimulating the bundle of His may be at 2 mm.

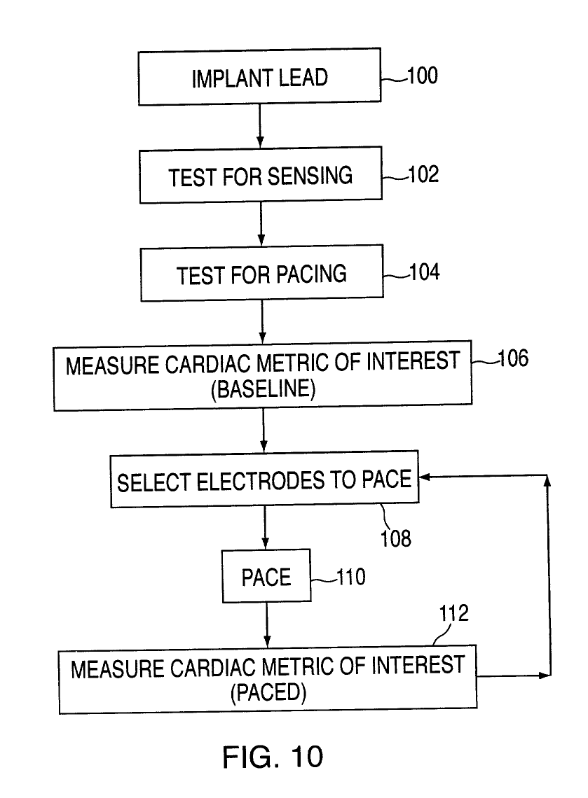

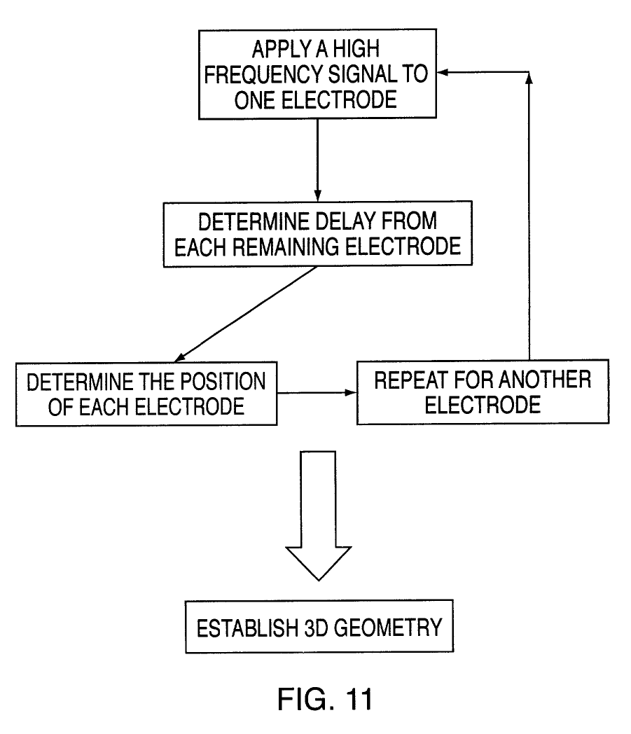

FIG. 10 shows a basic flow chart for selecting the electrodes. As can be seen in this flow chart, the process is adaptive to take advantage of the large number of electrodes that are available for sensing and pacing. The leads are implanted in step 100. In step 102 the electrodes are tested to determine which electrodes are most suitable for this purpose. This step can be done either using a brute force approach, i.e., by testing every electrode.

Alternatively, the test may be started with a group of electrodes that are known to be more suitable for sensing from statistical analysis, or from other empirical data.

The pacing electrodes are designated in a similar manner in step 104, using standard techniques known in the art. For example, a tachycardia is induced in the patient, and then antitachycardiac pacing pulses are applied to the electrodes in turn until the tachycardia is reverted. This process is used not only to detect the optimal pacing electrodes, but their thresholds as well. The round robin approach described above is a brute force method for optimizing pacing for CHF and it does not include the skill and experience of the attending physician. For example, since electrodes E15 and E16 are near the apex of the RV, the physician may decide that these two electrodes need not be tested. Likewise if 3D positioning determines E9 through E12 are the most likely to produce good results because they are below the moderator band of the RV, then testing only through those electrodes can be performed. Or, if the physician knows that the patient has dead tissue in a certain location due to a previous myocardial infarction, electrodes over the infracted tissue will not be utilized for this patient. This ‘intelligent’ approach means that much less data need to be collected for any given patient.

In co-application Ser. No.______ entitled Method and apparatus for Determining Spatial Relation of Multiple Impantable Electrodes filed Apr. 25, 2002, methods and techniques are disclosed for determining after implantation (step 100 in FIG. 10) the actual three-dimensional position of the lead and its electrodes within the respect cardiac chambers. This information is then utilized as part of determining how to select the sensing and pacing electrodes.

Preferably, after implantation, the positions of the various electrodes is determined within the cardiac chambers in 3D space. These positions are determined by sending a carrier signal from the pacemaker P through one of the electrodes and sensing that signal on all of the other electrodes. Using internal algorithms (described more fully in the above-identified commonly assigned application Ser. No. ______, entitled Method and apparatus for Determining Spatial Relation of Multiple Impantable Electrodes, incorporated herein by reference) the pulse generator then reconstructs the electrode position. Briefly sensing of intrinsic cardiac activities are performed through each electrodes. This determines which electrodes are in close proximity to wavelets characteristic of these activities.

Preferably, for each of the embodiments described above, the multi-electrode lead is preformed into the shape shown, for example, by making the tube 18 of a polymer or other shape retaining material. During implantation, the lead is straightened by using a stylet inserted into the tube, or by other means known in the art. After insertion, the stylet is removed allowing the lead to take its normal shape shown in the drawings.

Obviously numerous modifications may be made to the invention without departing from the invention as disclosed herein.

I claim:

1. An implantable cardiac device comprising:
a pulse generator; and
a lead having a proximal end and a distal end with a plurality of electrodes, wherein said lead is preformed to a predetermined shape selected to position said plurality of electrodes adjacent to a preselected cardiac tissue when implanted.

2. The device of claim 1 wherein said electrodes are spaced at a predetermined distance, and adapted to receive respective pacing pulses, said pacing pulses being selected to generate a waveform that propagates along the cardiac tissue in a manner that mimics a sinus wave form.

3. The device of claim 1 wherein said lead is shaped for implantation in the right ventricle with said electrodes being positioned along and in contact with the septal wall.

4. The device of claim 3 wherein said lead includes a distal tip and wherein said lead is shaped to position said tip at the RVOT.

5. The device of claim 4 wherein said plurality of electrodes include a first pacing electrode positioned in the vicinity of the optimal point for pacing the septal wall and additional pacing electrodes disposed below said first pacing electrode.

6. The device of claim 5 wherein said pacing electrodes are spaced from each other at a distance in the range of 0.3 to 5 mm.

7. The device of claim 5 wherein said pacing electrodes number between 4 and 32 electrodes.

8. The device of claim 4 wherein said lead includes a first portion and a second portion, said lead being shaped to position said first portion adjacent to the septal wall and said second portion adjacent to the free wall of the right ventricle.

9. The device of claim 8 wherein said lead is provided with electrodes on said second portion.

10. The device of claim 4 wherein said lead is V-shaped.

11. The device of claim 3 wherein said lead is spiral shaped.

12. The device of claim 1 1 wherein said lead includes a first set of electrodes positioned along the septal wall and a second set of electrodes positioned along the free wall of the right ventricle.

13. The device of claim 12 wherein said first and second set of electrodes receive pacing pulses and are arranged to generate a polarization waveshape mimicking the waveshape produced by the sinus Purkinje cell system.

14. The device of claim 1 wherein said lead includes a first portion adapted for positioning in the atrium and a second portion adapted to be positioned in the ventricle.

15. The device of claim 14 wherein said first portion includes a set of electrodes receiving pacing signals, said first portion being positioned to stimulate the His bundle with said pacing signals.

16. An implantable lead for a cardiac device comprising:
an elongated member having a predetermined shape and at least three electrodes disposed on said elongated member, said predetermined shape being selected to position said electrodes in contact with a predetermined tissue cardiac tissue when implanted.

17. The lead of claim 16 wherein said elongated member is preshaped to position said electrodes in the right ventricle.

18. The lead of claim 17 wherein said elongated member is preshaped to position said electrodes in contact with the septal wall.

19. The lead of claim 17 wherein said elongated member is terminated in a tip, said elongated member being shaped to position said tip at the RVOT.

20. The lead of claim 19 wherein said elongated member is V-shaped.

21. The lead of claim 16 wherein said elongated member is a spiral.

22. The lead of claim 21 wherein said elongated member is shaped and constructed for positioning in the ventricle.

23. The lead of claim 21 comprising a first set of electrodes disposed on said elongated member, wherein said elongated member is shaped to position said first set of electrodes in contact with the septal wall.

24. The lead of claim 23 further comprising a second set of electrodes disposed on said lead, wherein said elongated member being is shaped to position said second set of electrodes in contact with the free wall of the ventricle.

25. The lead of claim 16 wherein said elongated member includes a first portion, and a second portion, each portion being provided with at least three electrodes, wherein said elongated member is shaped to position said first portion in the atrium and the second portion in the ventricle.

26. The lead of claim 25 wherein said elongated member is shaped to position the electrodes of said first portion to stimulate the bundle of His.

27. The lead of claim 26 wherein said first portion includes a loop, with said elongated member being shaped to position said loop above the tricuspid valve.

28. The lead of claim 26 wherein said elongated member is shaped to position the electrodes of said second portion to stimulate the septal wall.

29. The lead of claim 28 wherein said second portion is terminated in a tip, said elongated member being shaped to position said tip at the RVOT.

30. A method of providing therapeutic pulses to treat CHF in a patient comprising the steps of:
implanting in a cardiac chamber a lead, said lead a plurality of electrodes, said lead being preformed into a predetermined shape;
deploying said lead in said cardiac chamber to said preselected shape to position said plurality of electrodes in contact with preselected cardiac tissues;
designating a first set of said plurality of electrodes as sensing electrodes;
designating a second set of said plurality of electrodes as pacing electrodes;
monitoring the heart through said sensing electrodes to sense intrinsic cardiac signals; and
pacing said cardiac tissues in response to said intrinsic cardiac signals.

31. The method of claim 30 further comprising deploying said lead in the ventricle.

32. The method of claim 31 further comprising deploying said lead to position said electrodes in contact with the septal wall.

33. The method of claim 32 further comprising pacing said septal wall starting from the optimal pacing point of the septal wall.

34. The method of claim 31 further comprising deploying said lead to position said electrodes in contact with the septal wall and the free wall of the ventricle.

35. The method of claim 31 further comprising deploying said lead to position said electrodes to pace the His bundle.
